(19) United States
(12) Patent Application Publication
(10) Pub. No.: US 2024/0020823 A1
(43) Pub. Date: Jan. 18, 2024

(54) ASSISTANCE DIAGNOSIS SYSTEM FOR LUNG DISEASE BASED ON DEEP LEARNING AND ASSISTANCE DIAGNOSIS METHOD THEREOF

(71) Applicants: DEEPNOID CO., LTD., Seoul (KR); GIMHAE BIOMEDICAL CENTER, Gimhae-si (KR); JEONGSEOK RESEARCH FOUNDATION, Gimhae-si (KR)

(72) Inventors: Tae-Gyu KIM, Yongin-si (KR); Hyun-Ju CHOI, Gimhae-si (KR); Hwa-Pyung KIM, Seoul (KR); Hao LI, Seoul (KR); Dae-Woo SEOK, Gimhae-si (KR); Seung-Hoon LEE, Busan (KR); Woo-Sik CHOI, Gimpo-si (KR); Seung-Hwan LEE, Busan (KR)

(21) Appl. No.: 17/293,250

(22) PCT Filed: Mar. 22, 2021
(86) PCT No.: PCT/KR2021/003481

§ 371 Date: Sep. 26, 2023

(30) Foreign Application Priority Data
Dec. 22, 2020 (KR) 10-2020-0180989

(51) Int. Cl. G06T 7/00; G06T 7/194; G06T 7/11; G06V 20/50; G06V 10/764; G06V 10/774; G16H 50/20; G16H 30/40; G16H 50/50

(52) U.S. Cl. CPC G06T 7/0012; G06T 7/194; G06T 7/11; G06V 20/50; G06V 10/764; G06V 10/774; G16H 50/20; G16H 30/40; G16H 50/50; G06T 2207/30008; G06T 2207/30096; G06T 2207/30061; G06T 2207/20081; G06V 2201/031; G06V 2201/033; G06T 2207/30204; G06T 2207/10116

(57) ABSTRACT

A deep learning-based lung disease diagnosis assistance system according to an embodiment of the present disclosure includes an image input unit inputting a diagnosis target image obtained by capturing a lung image; a bone area removal unit removing a bone area from the diagnosis target image to output a soft tissue image from which the bone area is removed, on the basis of the bone binary model; a lung area extraction unit extracting a lung area from the soft tissue image to output a lung image of the lung area on the basis of a lung segmentation model; and a lung disease diagnosis unit diagnosing whether lung disease is present in the lung image on the basis of a lung disease detection model.

### TECHNICAL FIELD

The present disclosure relates to a deep learning-based lung disease diagnosis assistance system and a deep learning-based lung disease diagnosis assistance method and, more particularly to a deep learning-based lung disease diagnosis assistance system and a deep learning-based lung disease diagnosis assistance method, which are capable of detecting lung disease from a diagnosis target image obtained by capturing a lung image of a subject to be diagnosed through the previously registered diagnosis model.

### BACKGROUND ART

In modern medicine, medical imaging is a very important tool for effective disease diagnosis and patient treatment. In addition, the development of imaging technology makes it possible to acquire more sophisticated medical imaging data. Such increasing sophistication results in increasing amounts of data, whereby there are many difficulties in analyzing medical imaging data due to limitations of human vision. Accordingly, in recent decades, clinical decision support systems and computer-assisted diagnostic systems have played an essential role in automatically analyzing medical images.

The clinical decision support systems or computer assistance diagnostic systems in the related art detects and marks a lesion site, or presents the diagnostic information to medical staff or medical practitioners (hereinafter referred to as users).

For example, “Medical image-based disease diagnosis information calculating method and apparatus” disclosed in Korean Patent Application Publication No. 10-2017-0017614 includes detecting areas of interest in which an object to be analyzed is photographed, calculating the variation coefficient, creating an image of the variation coefficient, and comparing the same to a reference sample, and thus has an effect of diagnosing the degree of a patient's disease by using medical images acquired through CT, MRI, and ultrasound imaging apparatuses.

In particular, in recent years, artificial intelligence (AI) technology based on machine learning such as deep learning contributes to bringing about a breakthrough in diagnosing a patient's disease using medical imaging.

Deep learning refers to a subset of machine learning based on an artificial neural network, which is obtained by simulating the human biological neuron, to allow the machine to learn. Recently, deep learning technology has rapidly developed in the field of image recognition, and has been widely used in the field of diagnosis of medical images.

In deep learning technology, a diagnostic model for diagnosing diseases is formed by repeatedly learning the training data. Since types of diseases used as learning data are varied, it is important to develop a diagnostic model specialized for each disease. This means that a diagnostic model that derives near-perfect diagnostic results for a specific disease can be also applied to other diseases.

The assistance diagnosis method using such deep learning technology can also be applied to lung diseases. In the case of thoracic and cardiovascular surgery in which there are various specialized fields, there may be cases where an external expert's help is requested in order to accurately determine a patient's disease.

Therefore, when a lung diseases assistance diagnosis technology capable of automatically identifying abnormal areas such as lung lesions is proposed, it may be widely used as an auxiliary in the field.

### DISCLOSURE

### Technical Problem

Accordingly, an objective of the present disclosure is to provide a deep learning-based lung disease diagnosis assistance system and a deep learning-based lung disease diagnosis assistance method, which are capable of detecting lung disease from a diagnosis target image obtained by capturing an image of a lung of a subject to be diagnosed through the previously registered diagnosis model.

Another objective of the present disclosure is to provide a deep learning-based lung disease diagnosis assistance system and a deep learning-based lung disease diagnosis assistance method, wherein bone areas, such as ribs, that cover lungs, are removed from the diagnosis target image, to increase the clarity of the soft tissue, whereby the accuracy of diagnosis can be improved when diagnosing lung diseases through the diagnostic model.

Another objective of the present disclosure is to provide a deep learning-based lung disease diagnosis assistance system and a deep learning-based lung disease diagnosis assistance method, in which lesion sites are visually marked on the diagnosis result image, so that visualization of the lesion sites can make it possible to assist a medical practitioner in making diagnostic decisions.

### Technical Solution

A deep learning-based lung disease diagnosis assistance system according to an embodiment of the present disclosure includes an image input unit inputting a diagnosis target image obtained by capturing a lung image; a bone area removal unit removing a bone area from the diagnosis target image to output a soft tissue image from which the bone area is removed, on the basis of the bone binary model; a lung area extraction unit extracting a lung area from the soft tissue image to output a lung image of the lung area on the basis of a lung segmentation model; and a lung disease diagnosis unit diagnosing whether lung disease is present in the lung image on the basis of a lung disease detection model.

Herein, when a plurality of lung images and lung disease information for each for the lung images are input as lung disease learning data, the lung disease detection model may be generated by performing deep learning on the lung disease learning data through a previously registered classification algorithm, the classification algorithm being an algorithm for classifying the lung image on a per disease type basis according to the lung disease information.

In addition, when a lesion site is detected in the lung image, the lung disease diagnosis unit may output a diagnosis result image in which the lesion site is marked on the lung image on the basis of the lung disease detection model.

When a plurality of chest images and a bone binary image in which each chest image is binary are input as bone area learning data, the bone binary model may be generated through deep learning with the bone area learning data as inputs, to output the bone binary image.

According to an embodiment of the present disclosure, the bone area removal unit may input the diagnosis target image and output the bone binary image on the basis of the bone binary model, and remove a part corresponding to the bone area of the bone binary image from the diagnosis target image as the bone binary image is overlaid on the diagnosis target image, to output the soft tissue image, on the basis of a previously registered area removal algorithm.

According to an embodiment of the present disclosure, when a plurality of soft tissue images and a lung segmentation image obtained by segmenting the lung area for each of the soft tissue images are input as lung area learning data, the lung segmentation model may be generated through deep learning with the lung area learning data as inputs, to output the lung segmentation image.

According to an embodiment of the present disclosure, the lung area extraction unit may input the soft tissue image and output the lung segmentation image on the basis of the lung segmentation model, and extract the lung area from the soft tissue image as the lung segmentation image is overlaid on the soft tissue image, to output the lung image, on the basis of a previously registered area extraction algorithm.

According to an embodiment of the present disclosure, the image input unit may pre-process the diagnosis target image through a previously registered image pre-processing algorithm.

A deep learning-based lung disease diagnosis assistance method according to an embodiment of the present disclosure includes (A) performing deep learning on lung disease to generate a diagnostic model using learning data; (B) inputting a diagnosis target image in which a lung image is captured; and (C) diagnosing whether lung disease is present in the diagnosis target image on the basis of the diagnostic model, wherein the performing includes: (A1) when a plurality of chest images and a bone binary image in which a bone area of each of the chest images are binary are input as bone area learning data, generating a bone binary model through deep learning with the bone area learning data as inputs; (A2) when a plurality of soft tissue images and a lung segmentation image obtained by segmenting a lung area for each of the soft tissue images are input as lung area learning data, generating a lung segmentation model through deep learning with the lung area learning data as inputs; and (A3) when a plurality of lung images and lung disease information for each of the lung images are input as lung disease learning data, generating a lung disease detection model through deep learning with the lung disease learning data as inputs, wherein in the diagnosing, the bone binary model, the lung segmentation model, and the lung disease detection model are applied as the diagnostic model.

Herein, the diagnosing may include (C1) outputting the diagnosis target image as the bone binary image on the basis of the bone binary model; (C2) overlaying the bone binary image on the diagnosis target image; (C3) removing the bone area of the bone binary image from the diagnosis target image to output the soft tissue image on the basis of a previously registered area removal algorithm; (C4) outputting the soft tissue image as the lung segmentation image on the basis of the lung segmentation model; (C5) overlaying the lung segmentation image on the soft tissue image; (C6) extracting the lung area from the soft tissue image to output the lung image for the lung area on the basis of a previously registered area extraction algorithm; and (C7) detecting whether a lesion site is present in the lung image on the basis of the lung disease detection model.

In addition, the diagnosing may further include C8) when the lesion site is detected in the lung image, outputting a diagnosis result image in which the lesion site is marked on the lung image.

In the inputting, the diagnosis target image may be pre-processed through a previously registered image pre-processing algorithm.

According to an embodiment of the present disclosure, when the lung disease learning data is input as input data, the lung disease detection model may be generated by performing deep learning on the lung disease learning data through a previously registered classification algorithm, the classification algorithm being an algorithm for classifying the lung image on a per disease type basis according to the lung disease information.

### Advantageous Effects

According to the present disclosure, it is possible to detect lung disease from a diagnosis target image obtained by capturing an image of a lung of a subject to be diagnosed through a previously registered diagnosis model.

The present disclosure has effects of removing bone area, such as ribs, that covers lungs from the diagnosis target image to improve the clarity of a soft tissue image, and extracting a lung area from the soft tissue image and thus creating a lung image to improve the clarity of the lung area.

According to the present disclosure, it is possible to apply a lung image, from which unnecessary elements (rib and other organs such as the heart and liver) are removed, to a diagnostic model, when diagnosing lung diseases, thereby improving the accuracy of diagnosis.

According to the present disclosure, it is possible to visually mark the lesion site on the diagnosis result image, whereby visualization of the lesion area makes it possible to assist a medical practitioner in making diagnostic decisions.

### DESCRIPTION OF DRAWINGS

FIG. 1 schematically illustrates a configuration diagram of a deep learning-based lung disease diagnosis assist system according to an embodiment of the present disclosure;

FIG. 2 is a diagram illustrating a bone binary model according to an embodiment of the present disclosure;

FIG. 3 is a diagram illustrating a lung segmentation model according to an embodiment of the present disclosure;

FIG. 4 is a diagram illustrating a lung disease detection model according to an embodiment of the present disclosure;

FIG. 5 is a diagram illustrating an image processing process in a bone area removal unit according to an embodiment of the present disclosure;

FIG. 6 is a diagram illustrating an image processing process in a lung area extraction unit according to an embodiment of the present disclosure;

FIG. 7 is a flow chart illustrating a deep learning-based lung disease diagnosis assistance method according to an embodiment of the present disclosure;

FIGS. 8 and 9 are diagrams illustrating generation of a deep learning-based diagnostic model according to an embodiment of the present disclosure; and

FIG. 10 is a diagram illustrating a process in which lung disease is diagnosed from a diagnosis subject image through the diagnostic model according to an embodiment of the present disclosure.

### BEST MODE

A deep learning-based lung disease diagnosis assistance system according to an embodiment of the present disclosure includes an image input unit inputting a diagnosis target image obtained by capturing a lung image; a bone area removal unit removing a bone area from the diagnosis target image to output a soft tissue image from which the bone area is removed, on the basis of the bone binary model; a lung area extraction unit extracting a lung area from the soft tissue image to output a lung image of the lung area on the basis of a lung segmentation model; and a lung disease diagnosis unit diagnosing whether lung disease is present in the lung image on the basis of a lung disease detection model.

Mode for Disclosure

Hereinafter, referring to accompanying drawings, a deep learning-based lung disease diagnosis assistance system and a deep learning-based lung disease diagnosis assistance method according to preferable embodiments of the present disclosure will be described.
Deep learning-based lung disease diagnosis assistance system

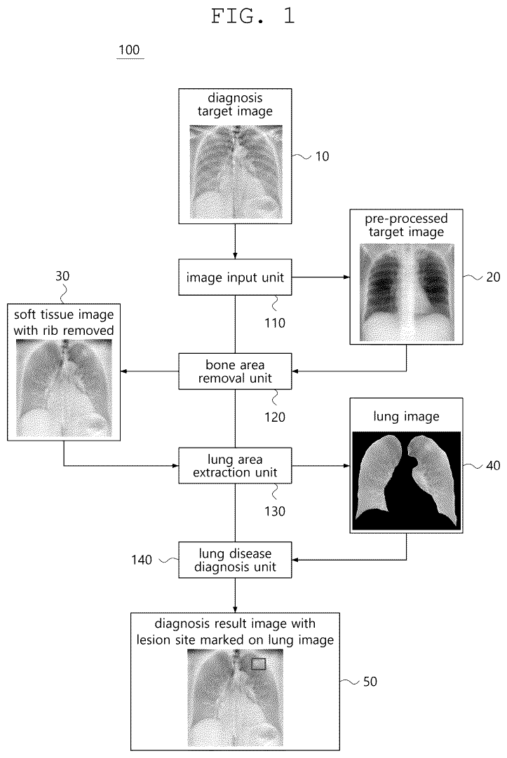

Hereinafter, a deep learning-based lung disease diagnosis assistance system will be described with reference to FIGS. 1 to 6.

Referring to FIG. 1, the deep learning-based lung disease diagnosis assistance system 100 may include an image input unit 110, a bone area removal unit 120, a lung area extraction unit 130, and a lung disease diagnosis unit 140.

A diagnosis target image 10 is input to the image input unit 110. Here, the diagnosis target image 10 may be a chest image in which a lung image is captured, in which the target is specified. The chest image may be an X-ray image. The image input unit 110 pre-processes the diagnosis target image 10 through a previously registered image pre-processing algorithm, and outputs the same as a pre-processed image 20.

According to the embodiment, a bone binary model is previously registered in the bone area removal unit 120, a lung segmentation model 135 is previously registered in the lung area extraction unit 130, and a lung disease detection model 145 is previously registered in the lung disease diagnosis unit 140.

Here, the bone binary model 125, the lung segmentation model 135, and the lung disease detection model 145 may be generated through deep learning with learning data as inputs.

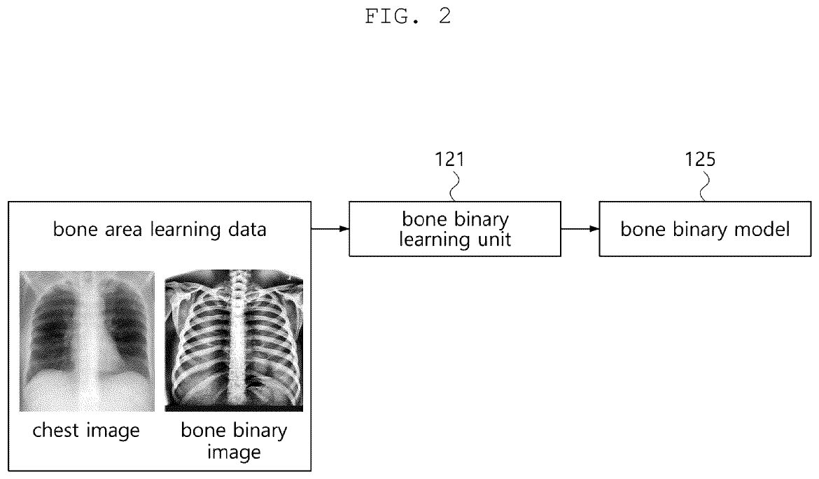

Referring to FIG. 2, when bone area learning data is input to the bone binary learning unit 121, the bone binary model 125 is generated by performing deep learning on the bone area in the chest image.

The bone area learning data may include a plurality of chest images and a bone binary image 25 in which each chest image is binary.

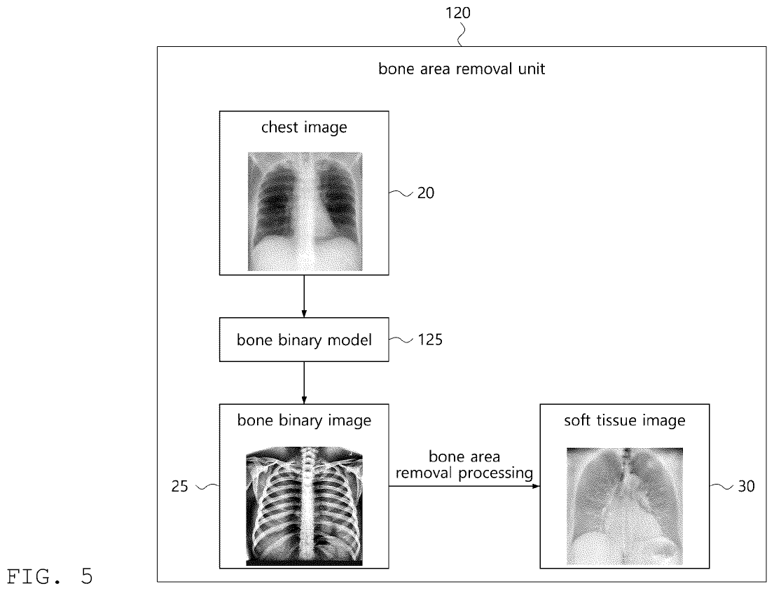

Referring to FIG. 5, the bone binary model 125 inputs a chest image of the diagnosis target image 10 and outputs the bone binary image 25.

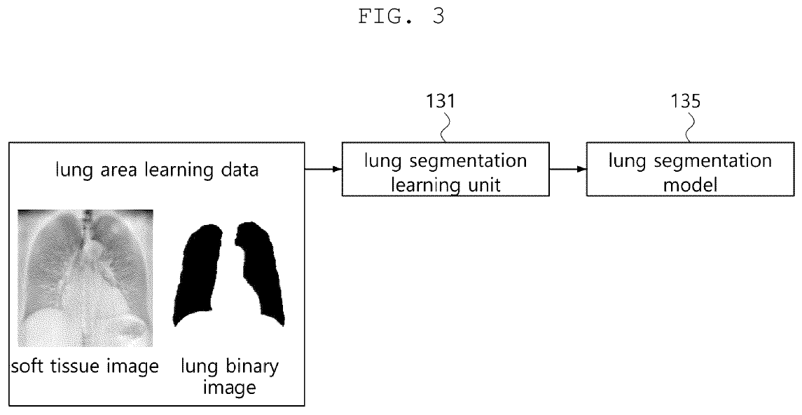

Referring to FIG. 3, when the lung area learning data is input to the lung segmentation learning unit 131, the lung segmentation model 135 is generated by performing deep learning on the lung area in a soft tissue image 30.

The lung area learning data may include a plurality of soft tissue images 30 and a lung segmentation image 35 obtained by segmenting the lung area for each soft tissue image 30.

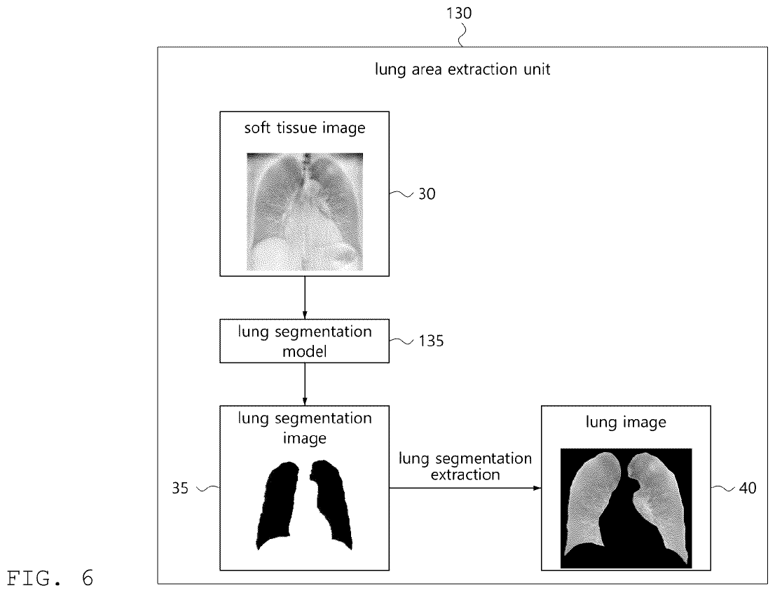

Referring to FIG. 6, the lung segmentation model 135 inputs the soft tissue image and outputs the lung segmentation image 35 obtained by segmenting the lung area in the soft tissue image 30.

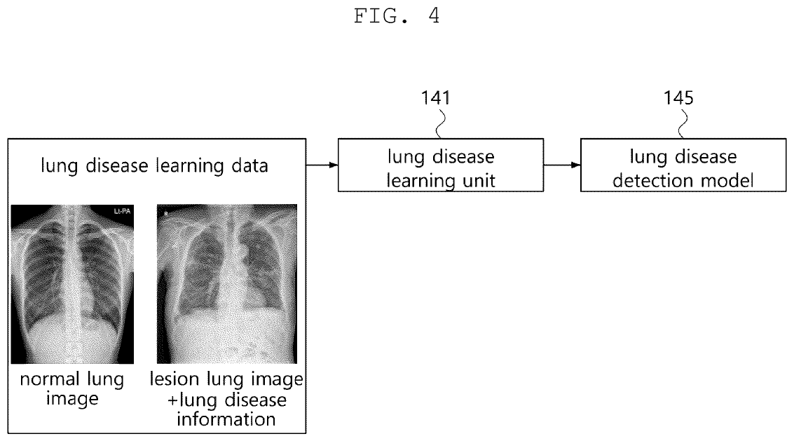

Referring to FIG. 4, when lung disease learning data is input to the lung disease learning unit, the lung disease detection model 145 is generated by performing deep learning on the lung disease learning data through a previously registered classification algorithm. Here, the classification algorithm is an algorithm for classifying a lung image 40 on a per-disease type basis according to lung disease information.

The lung disease learning data may include a plurality of lung images 40 and lung disease information for each lung image 40. Here, the lung image 40 may include a normal lung image 40 without a lung lesion and a lesion lung image 40 with a lung lesion. The lung disease information is information on normal, pneumothorax, tuberculosis, pneumonia, lung cancer, and the like.

Referring to FIGS. 1 and 5, when the diagnosis target image 10 is input, the bone area removal unit 120 outputs a soft tissue image 30 from which the bone area is removed from the diagnosis target image 10.

First, the bone area removal unit 120 outputs the pre-processed image 20 as the bone binary image 25, on the basis of the bone binary model 125. The bone binary image 25 may be an image in which the bone area of the pre-processed image 20 and a portion other than the bone area are binary in black and white. In addition, the soft tissue image 30 may be an image in which only soft tissues (lung, heart, liver, etc.) exist after the bone area is removed from the pre-processed image 20.

Subsequently, since the diagnosis target image 10 is overlaid on the bone binary image 25, the bone area removal unit 120 removes a part corresponding to the bone area of the bone binary image 25 from the diagnosis target image 10, on the basis of an area removal algorithm that is previously registered. Here, the area removal algorithm may be an image processing algorithm that removes the part corresponding to the bone area of the bone binary image 25 from the diagnosis target image 10.

Referring to FIGS. 1 and 6, when the soft tissue image 30 is input, the lung area extraction unit 130 separately extracts only the lung area from the soft tissue image 30 to output the lung image 40 for the lung area.

The lung area extraction unit 130 outputs a lung segmentation image 35 obtained by segmenting the lung area from the soft tissue image, on the basis of the lung segmentation model.

Subsequently, as the soft tissue image 30 is overlaid on the lung segmentation images 35, the lung area extraction unit 130 extracts the lung area from the soft tissue image 30 to output the lung image 40 on the basis of an area extraction algorithm that is previously registered.

Here, the area extraction algorithm may be an image processing algorithm for extracting a part corresponding to the lung area of the lung segmentation image 35 from the soft tissue image 30. In addition, the lung image 40 may be an image in which only the lung area is present after removing parts other than the lung area from the soft tissue image 30.

Referring to FIG. 1, the lung disease diagnosis unit 140 diagnoses whether a lung disease is present in a lung image 40, on the basis of the lung disease detection model 145. In addition, when the lesion site is detected from the lung image 40, the lung disease diagnosis unit 140 outputs a diagnosis result image 50 in which the lesion site 51 is marked on the lung image 40, on the basis of the lung disease detection model 145.
Deep learning-based lung disease diagnosis assistance method

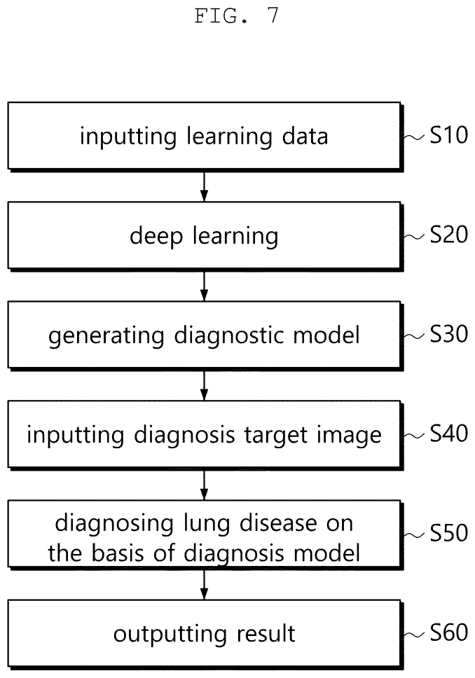

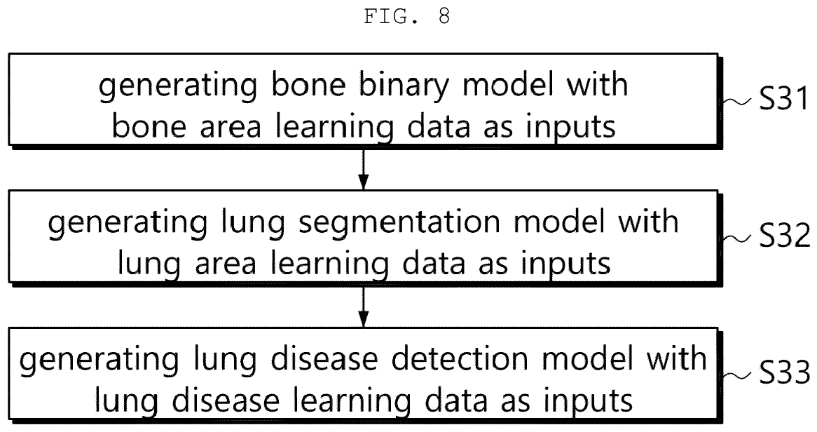

Hereinafter, with reference to FIGS. 7 to 10, a deep learning-based lung disease diagnosis assistance method according to an embodiment of the present disclosure will be described.

The learning data is input as input data (S10), and a diagnostic model may be generated by performing deep learning on the lung disease (S30). As a diagnostic model, a bone binary model 125, a lung segmentation model 135, and a lung disease detection model 145 may be generated.

The bone binary model may be generated through deep learning with bone area learning data as inputs (S31). The bone area learning data may include a plurality of chest images, and a bone binary image 25 in which the bone area for each chest image is binary.

The lung segmentation model 135 is generated through deep learning with the lung area learning data as inputs (S32). The lung area learning data may include a plurality of soft tissue images 30 and a lung segmentation image 35 obtained by segmenting the lung area for each soft tissue image 30.

When lung disease learning data is input, the lung disease detection model 145 is generated by performing deep learning on the lung disease learning data through a classification algorithm that is previously registered (S33). The lung disease learning data may include a plurality of lung images 40 and lung disease information for each lung image 40. The classification algorithm may be an algorithm for classifying the lung image 40 on a per disease type basis (normal, pneumothorax, tuberculosis, asthma, cancer, etc.) according to lung disease information.

A diagnosis target image 10 obtained by capturing a lung image is input as a diagnostic model (S40). The diagnosis target image 10 may be previously processed through an image pre-processing algorithm that is previously registered.

When the diagnosis target image 10 is input, it is diagnosed whether lung disease is present in the diagnosis target image 10 on the basis of the diagnosis model (S50). As described above, as the diagnostic model, the bone binary model 125, the lung segmentation model 135, and the lung disease detection model 145 are applied.

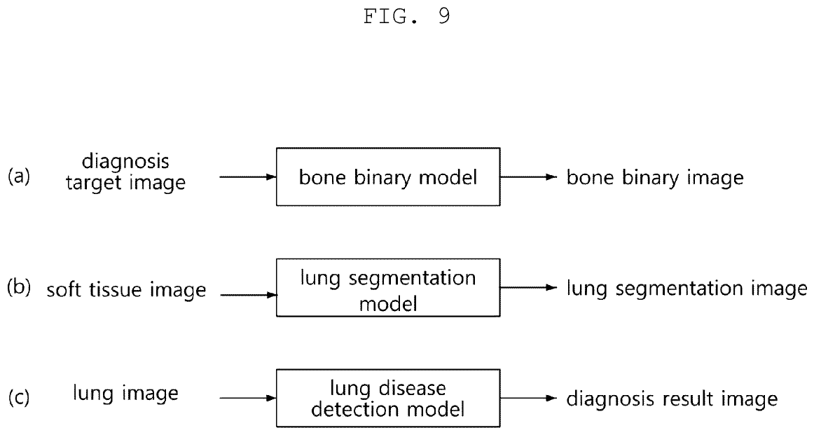

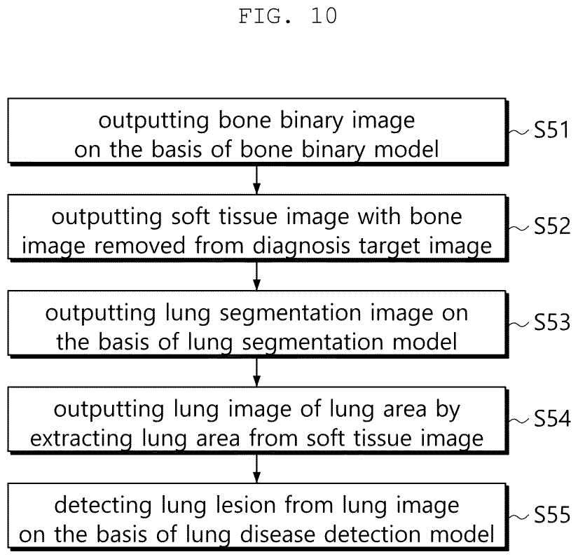

Referring to FIGS. 9(a) and 10, the diagnosis target image 10 is output as a bone binary image 25 on the basis of the bone binary model 125 (S51).

The bone binary image 25 is overlaid on the diagnosis target image 10. Then, the bone area of the bone binary image 25 is removed from the diagnosis target image on the basis of the previously registered area removal algorithm, to output a soft tissue image 30 (S52).

Referring to FIGS. 9(b) and 10, the soft tissue image 30 is output as a lung segmentation image 35 through the lung segmentation model 135 (S53).

The lung segmentation image 35 is overlaid on the soft tissue image 30. Then, the lung area is extracted from the soft tissue image 30 to output the lung image 40 of the lung area on the basis of the previously registered area extraction algorithm (S54).

Referring to FIGS. 9(b) and 10, the lung image 40 is output as a diagnosis result image on the basis of the lung disease detection model 145 (S55). The diagnosis result image may be an image in which a lesion site is marked on the lung image 40, and a diagnosis name for the disease is also output (S60).

According to the present disclosure, bone areas, such as ribs, that covers lungs, are removed from the diagnosis target image 10, thereby increasing the clarity of the soft tissue, and the lung area is extracted from the soft tissue image 30 to generate the lung image 40, thereby improving the clarity of the lung area.

According to the present disclosure, it is possible to detect lung disease from the diagnosis target image 10 in which the lung of a subject to be diagnosed is captured through the previously registered diagnosis model.

The present disclosure has an effect of improving the accuracy of diagnosis by applying, to the diagnostic model, the lung image 40 from which unnecessary elements (ribs and other organs such as the heart and liver) are removed when diagnosing lung disease.

According to the present disclosure, since the lesion site is visually marked on the diagnosis result image, visualization of the lesion site can make it possible to assist a medical practitioner in making diagnostic decisions.

Although several embodiments of the present disclosure have been shown and described, it will be apparent to those skilled in the art to which the present disclosure pertains that modifications can be made to the present embodiment without departing from the spirit or spirit of the present disclosure. The scope of the disclosure will be defined by the appended claims and their equivalents.

### Industrial Usability

The present disclosure can be applied to assist in the diagnosis of lung disease based on deep learning technology.

1. A deep learning-based lung disease diagnosis assistance system, comprising:
an image input unit inputting a diagnosis target image obtained by capturing a lung image;
a bone area removal unit removing a bone area from the diagnosis target image to output a soft tissue image from which the bone area is removed, on the basis of the bone binary model;
a lung area extraction unit extracting a lung area from the soft tissue image to output a lung image of the lung area on the basis of a lung segmentation model; and
a lung disease diagnosis unit diagnosing whether lung disease is present in the lung image on the basis of a lung disease detection model.

2. The system of claim 1, wherein when a plurality of lung images and lung disease information for each for the lung images are input as lung disease learning data, the lung disease detection model is generated by performing deep learning on the lung disease learning data through a previously registered classification algorithm, the classification algorithm being an algorithm for classifying the lung image on a per disease type basis according to the lung disease information.

3. The system of claim 2, wherein when a lesion site is detected in the lung image, the lung disease diagnosis unit outputs a diagnosis result image in which the lesion site is marked on the lung image on the basis of the lung disease detection model.

4. The system of claim 1, wherein when a plurality of chest images and a bone binary image in which each chest image is binary are input as bone area learning data, the bone binary model is generated through deep learning with the bone area learning data as inputs, to output the bone binary image.

5. The system of claim 4, wherein the bone area removal unit inputs the diagnosis target image and outputs the bone binary image on the basis of the bone binary model, and removes a part corresponding to the bone area of the bone binary image from the diagnosis target image as the bone binary image is overlaid on the diagnosis target image, to output the soft tissue image, on the basis of a previously registered area removal algorithm.

6. The system of claim 1, wherein when a plurality of soft tissue images and a lung segmentation image obtained by segmenting the lung area for each of the soft tissue images are input as lung area learning data, the lung segmentation model is generated through deep learning with the lung area learning data as inputs, to output the lung segmentation image.

7. The system of claim 6, wherein the lung area extraction unit inputs the soft tissue image and outputs the lung segmentation image on the basis of the lung segmentation model, and extracts the lung area from the soft tissue image as the lung segmentation image is overlaid on the soft tissue image, to output the lung image, on the basis of a previously registered area extraction algorithm.

8. The system of claim 1, wherein the image input unit pre-processes the diagnosis target image through a previously registered image pre-processing algorithm.

9. A deep learning-based lung disease diagnosis assistance method, comprising:
(A) performing deep learning on lung disease to generate a diagnostic model using learning data;
(B) inputting a diagnosis target image in which a lung image is captured; and
(C) diagnosing whether lung disease is present in the diagnosis target image on the basis of the diagnostic model,
wherein the performing includes:
(A1) when a plurality of chest images and a bone binary image in which a bone area of each of the chest images are binary are input as bone area learning data, generating a bone binary model through deep learning with the bone area learning data as inputs;
(A2) when a plurality of soft tissue images and a lung segmentation image obtained by segmenting a lung area for each of the soft tissue images are input as lung area learning data, generating a lung segmentation model through deep learning with the lung area learning data as inputs; and
(A3) when a plurality of lung images and lung disease information for each of the lung images are input as lung disease learning data, generating a lung disease detection model through deep learning with the lung disease learning data as inputs,
wherein in the diagnosing, the bone binary model, the lung segmentation model, and the lung disease detection model are applied as the diagnostic model.

10. The method of claim 9, wherein the diagnosing comprises:
(C1) outputting the diagnosis target image as the bone binary image on the basis of the bone binary model;
(C2) overlaying the bone binary image on the diagnosis target image;
(C3) removing the bone area of the bone binary image from the diagnosis target image to output the soft tissue image on the basis of a previously registered area removal algorithm;
(C4) outputting the soft tissue image as the lung segmentation image on the basis of the lung segmentation model;
(C5) overlaying the lung segmentation image on the soft tissue image;
(C6) extracting the lung area from the soft tissue image to output the lung image for the lung area on the basis of a previously registered area extraction algorithm; and
(C7) detecting whether a lesion site is present in the lung image on the basis of the lung disease detection model.

11. The method of claim 10, wherein the diagnosing further comprises:
C8) when the lesion site is detected in the lung image, outputting a diagnosis result image in which the lesion site is marked on the lung image.

12. The method of claim 9, wherein in the inputting, the diagnosis target image is pre-processed through a previously registered image pre-processing algorithm.

13. The method of claim 9, wherein when the lung disease learning data is input as input data, the lung disease detection model is generated by performing deep learning on the lung disease learning data through a previously registered classification algorithm, the classification algorithm being an algorithm for classifying the lung image on a per disease type basis according to the lung disease information.
